(19) United States
(12) Patent Application Publication
(10) Pub. No.: US 2025/0127638 A1
(43) Pub. Date: Apr. 24, 2025

(54) STENT AND METHOD FOR SECURING AIR FLOW BY RELIEVING STENOSIS OF RESPIRATORY ORGAN

(71) Applicants: TORAY INDUSTRIES, INC., Tokyo (JP); REGENTS OF THE UNIVERSITY OF MINNESOTA, Minneapolis, MN (US)

(72) Inventors: Koji KADOWAKI, Kanagawa (JP); Roy Joseph CHO, Edina, MN (US); Daniel E. GLUMAC, Lino Lakes, MN (US); Ryan Coulson HUNTER, Little Canada, MN (US); Gregory Kermit PETERSON, Centerville, MN (US)

(73) Assignees: TORAY INDUSTRIES, INC., Tokyo (JP); REGENTS OF THE UNIVERSITY OF MINNESOTA, Minneapolis, MN (US)

(21) Appl. No.: 18/696,138

(22) PCT Filed: Sep. 27, 2022
(86) PCT No.: PCT/US2022/044812

§ 371 Date: Mar. 27, 2024

Related U.S. Application Data
(60) Provisional application No. 63/249,264, filed on Sep. 28, 2021.

(51) Int. Cl. A61F 2/82

(52) U.S. Cl. CPC A61F 2/82; A61F 2210/0009; A61F 2210/0076; A61F 2230/0069; A61F 2250/0056

(57) ABSTRACT

A stent for a respiratory organ, the stent having an inside surface and an outside surface, includes a base member and a hydrophilic polymer layer. The hydrophilic polymer layer contains a hydrophilic polymer having a hydroxy group and an amide group. The hydrophilic polymer layer is provided on at least a part of the inside surface and/or on at least a part of the outside surface.

### TECHNICAL FIELD

The present disclosure relates to a stent that can inhibit sticking of mucus and can also inhibit loss of cilia and excessive proliferation of goblet cells and hence is high in biocompatibility and a method for securing air flow by relieving stenosis of a respiratory organ using the stent.

### BACKGROUND ART

Stents are implant medical devices that can be left in bodies and some stents can be expanded in the radial direction. Stents are set inside various body cavities or vascular ducts (vascular system, esophagus, gastrointestinal tract, colon and small intestine, bile duct, pancreatic duct, lung pipes, ureter, nasal cavities and respiratory tract, trachea, bronchi, etc.). When a body cavity or a vascular duct is constricted, a stent is set inside a constricted portion to secure an inner cavity.

Among such stents are ones that are left in a body cavity or a vascular duct for a long period of time and ones that are removed from a body after they have served to keep an inner cavity open for a prescribed time.

For example, Non-patent literature 1 discloses a stent for respiratory tract that is left in a constricted part to enable breathing when the respiratory tract or bronchi are constricted by lung cancer or the like.

However, there are serious problems, that is, sticking of mucus, and loss of cilia and occurrence of complications such as excessive proliferation of goblet cells due to low biocompatibility. Thus, there are clinical needs for medical treatment using a stent that can be used for a long period of time while inhibiting occurrence of complications by inhibiting sticking of mucus and improving its biocompatibility.

To meet these needs, stents for respiratory tract that were coated with a hydrophilic polymer or a superhydrophobic polymer were developed (Patent literature 1 and Non-patent literature 1). Patent literature 2 discloses a device having a hydrophilic surface and a method for easily producing the same.

### CITATION LIST

### Patent Literature

[Patent literature 1] US 2017/0340782
[Patent literature 2] WO 2017/146102

### Non-Patent Literature

[Non-patent literature 1] Hans J. Lee et al., Journal of Thoracic Disease 2017; 9 (11): 4,651-4,659.

### SUMMARY

### Technical Problems

However, stents in related arts are insufficient in performance and have many problems to be solved.

In view of the above, an object of the present disclosure is to provide a stent for a respiratory organ that can inhibit sticking of mucus and occurrence of complications and is high in biocompatibility.

### Solution to Problem

To attain the above object, illustrative aspects of the present disclosure are as follows:
[1] A stent for a respiratory organ, the stent having an inside surface and an outside surface, in which: the stent includes a base member and a hydrophilic polymer layer; the hydrophilic polymer layer contains a hydrophilic polymer having a hydroxy group and an amide group; and the hydrophilic polymer layer is provided on at least a part of the inside surface.
[2] A stent for a respiratory organ, the stent having an inside surface and an outside surface, in which: the stent includes a base member and a hydrophilic polymer layer; the hydrophilic polymer layer contains a hydrophilic polymer having a hydroxy group and an amide group; and the hydrophilic polymer layer is provided on at least a part of the outside surface.
[3] The stent according to item [1] or [2], in which the base member contains a silicone resin.
[4] The stent according to any one of items [1] to [3], including a mixed layer of a component of the base member and a component of the hydrophilic polymer layer, the mixed layer being disposed between the base member and the hydrophilic polymer layer.
[5] The stent according to any one of items [1] to [4], in which the ratio X:Y between a thickness X of a layer including the hydrophilic polymer and a thickness Y of the base member is within a range of 1:400 to 1:120,000.
[6] The stent according to any one of items [1] to [5], including a tubular structure portion.
[7] The stent according to item [6], in which the tubular structure portion has an outer diameter of 4 mm or larger and 24 mm or smaller and has a thickness of 0.2 mm or larger and 2 mm or smaller.
[8] The stent according to any one of items [1] to [7], including plural projections or projections and recesses on the outside surface.
[9] The stent according to any one of items [1] to [8], in which the respiratory organ is a trachea, bronchus, or lung.
[10] The stent according to any one of items [1] to [9], in which the hydrophilic polymer having a hydroxy group and an amide group is at least one polymer selected from the group consisting of polyamides having a carboxyl group and copolymers of a monomer having a hydroxy group and a monomer having an amide group.
[11] The stent according to item [10], in which the monomer having a hydroxy group is at least one monomer selected from the group consisting of methacrylic acid, acrylic acid, vinylbenzoic acid, thiophen-3-acetic acid, 4-styrenesulphonic acid, vinylsulphonic acid, 2-acrylamide-2-methylpropane sulfonic acid, and their salts.
[12] The stent according to item or [11], wherein the monomer having an amide group is at least one monomer selected from the group consisting of N-vinylpyrrolidone, N-vinylacetamide, N,N-dimethylacrylamide, N,N-diethylacrylamide, N-isopropylacrylamide, N-(2-hydroxyethyl)acrylamide, and acrylamide.
[13] A method for securing air flow by relieving stenosis of a respiratory organ using a stent for a respiratory organ, the stent having an inside surface and an outside surface, in which: the stent includes a base member and a hydrophilic polymer layer; the hydrophilic polymer layer contains a hydrophilic polymer having a hydroxy group and an amide group; and the hydrophilic polymer layer is provided on at least a part of the inside surface.
[14] A method for securing air flow by relieving stenosis of a respiratory organ using a stent for a respiratory organ, the stent having an inside surface and an outside surface, in which: the stent includes a base member and a hydrophilic polymer layer; the hydrophilic polymer layer contains a hydrophilic polymer having a hydroxy group and an amide group; and the hydrophilic polymer layer is provided on at least a part of the outside surface.

Illustrative aspects of the present disclosure can provide a stent for a respiratory organ that can inhibit sticking of mucus is high in biocompatibility and thus inhibit occurrence of complications.

### BRIEF DESCRIPTION OF DRAWINGS

FIG. 1 is a schematic view of a specific stent according to one embodiment;

FIG. 2 illustrates an A-A cross section of the stent according to one embodiment shown in FIG. 1;

FIG. 3 is a schematic view of another specific stent according to one embodiment;

FIG. 4 is a schematic view of further specific stent according to one embodiment;

FIG. 5 is a schematic view of a stent that was used in Examples of the present disclosure; and

FIG. 6 is a schematic view of further specific stent according to one embodiment.

### DESCRIPTION OF EMBODIMENTS

A stent according to an embodiment (first embodiment) is a stent for a respiratory organ, having an inside surface and an outside surface, in which the stent includes a base member and a hydrophilic polymer layer, the hydrophilic polymer layer contains a hydrophilic polymer having a hydroxy group and an amide group and the hydrophilic polymer layer is provided on at least a part of the inside surface.

A stent according to another embodiment (second embodiment) is a stent for a respiratory organ, having an inside surface and an outside surface, in which the stent includes a base member and a hydrophilic polymer layer, the hydrophilic polymer layer contains a hydrophilic polymer having a hydroxy group and an amide group and the hydrophilic polymer layer is provided on at least a part of the outside surface.

The term “respiratory organ” as used in the disclosure is a generic term of organs relating to breathing and examples of them include a respiratory tract, oral cavity, nasal tracts, pharynx, trachea, bronchi, bronchioles, and lungs. The stent according to the embodiment is a stent for a respiratory organ and is preferably a stent for the respiratory tract, trachea, bronchus, or lung.

By setting and leaving the stent according to the embodiment in a constricted respiratory organ, stenosis of the respiratory organ is relieved so that air flow can be secured. The stent according to the embodiment can be applied to not only a constricted respiratory organ but also a clogged respiratory organ.

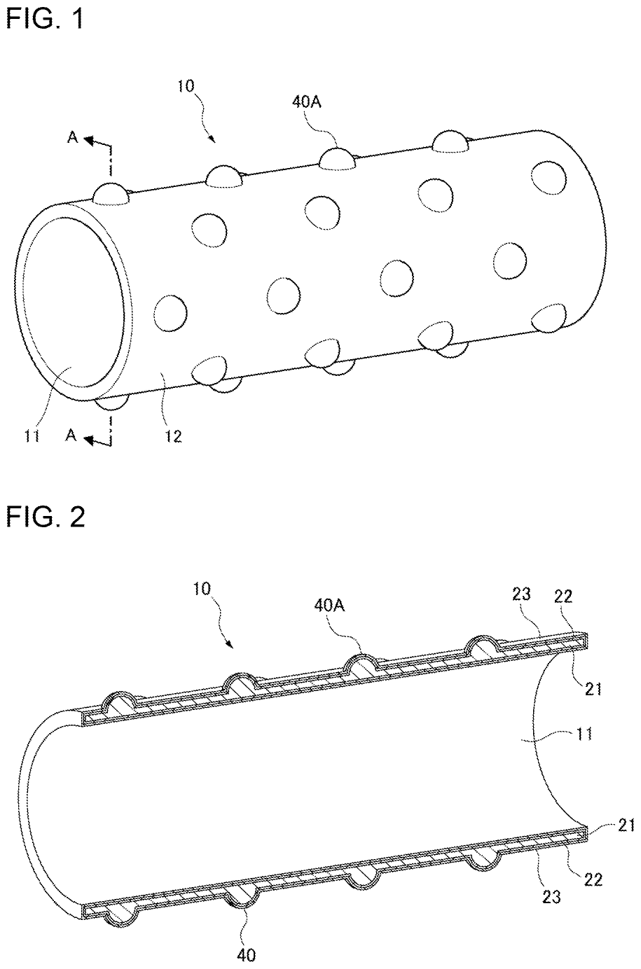

In the stent according to the embodiment, the term “inside surface” means a surface located on the side of passage of breathing air. The term “outside surface” means a surface other than the inside surface, that is, a surface located on the side of contact to a respiratory organ when the stent is applied to a living body. FIG. 1 is a schematic view of a stent 10 according to one embodiment. As illustrated in FIG. 1, the stent 10 according to the embodiment may include a tubular structure portion. In the stent 10 illustrated in FIG. 1, the inside surface of the tubular structure portion is an inside surface 11 and the surface other than the inside surface is an outside surface 12. The tubular structure portion may be capable of expanding in the radial direction.

### <Base Member>

The stent according to the embodiment includes a base member. There are no particular limitations on the material of the base member of the stent; the material may include a metal or a resin.

Examples of the metal include stainless steel, a cobalt alloy, a titanium alloy, and a nickel titanium alloy (Nitinol).

Examples of the resin include polyurethane, polyester, PTFE (polytetrafluoroethylene), and silicone resin. The use of silicone resin is preferable from the viewpoints of biocompatibility, physical properties relating to dynamics, workability, etc.

That is, it is preferable that the base member of the stent according to the embodiment contain a silicone resin.

The base member may be made of a single kind of material or two or more kinds of materials.

### <Hydrophilic Polymer Layer>

In the embodiment, the hydrophilic polymer layer provided on the surface(s) of the stent is a hydrophilic polymer formed on the surface(s) of the base member as a layer.

It is preferable that the stent according to the embodiment include, between the base member and the hydrophilic polymer layer, a mixed layer of the component(s) of the base member and the component(s) of the hydrophilic polymer layer.

In this specification, the hydrophilic polymer layer made only of a hydrophilic polymer and the mixed layer may be together referred to generically as a “layer including a hydrophilic polymer.”

FIG. 2 is an A-A cross section of the stent 10 according to the embodiment illustrated in FIG. 1.

For example, as illustrated in FIG. 2, it is preferable that the stent 10 according to the embodiment include, between a base member 21 and a hydrophilic polymer layer 23, a mixed layer 22 in which the component(s) of the base member 21 and the component(s) of the hydrophilic polymer layer 23 are mixed together.

The mixed layer disposed between the base member and the hydrophilic polymer layer may be formed by a part of the hydrophilic polymer constituting the hydrophilic polymer layer going into the base member. Alternatively, the mixed layer may be formed by a part of the base member going into the hydrophilic polymer layer. In the case where the stent according to the embodiment includes a mixed layer, the layer including a hydrophilic polymer has a layered structure of two or more layers including the hydrophilic polymer layer and the mixed layer.

In the stent according to the embodiment (first embodiment), the hydrophilic polymer layer needs to be provided on at least a part of the inside surface. The hydrophilic polymer may be provided on the entire inside surface. In the stent according to this embodiment, the hydrophilic polymer layer may be provided on at least a part of the outside surface in addition to the inside surface. In this case, the hydrophilic polymer may be provided on the entire outside surface in addition to the inside surface.

In the stent according to another embodiment (second embodiment), the hydrophilic polymer layer needs to be provided on at least a part of the outside surface. The hydrophilic polymer may be provided on the entire outside surface. In the stent according to this embodiment, the hydrophilic polymer layer may be provided on at least a part of the inside surface in addition to the outside surface. In this case, the hydrophilic polymer may be provided on the entire inside surface in addition to the outside surface.

That is, the hydrophilic polymer layer needs to be provided on at least a part of the inside surface and/or on at least a part of the outside surface. From the viewpoint of biocompatibility, the stent according to the embodiment is preferably provided with a hydrophilic polymer layer on all of the inside surface and the outside surface, that is, the entire surface of the stent. In the following description, the first embodiment will be described in detail. However, the descriptions for the first embodiment except for the location of the hydrophilic polymer layer can be applied to the second embodiment.

In the embodiment, since the hydrophilic polymer layer is provided on the surface of the stent, hydrophilicity is imparted to at least a part of the surface of the stent. Usually, the material of the hydrophilic polymer layer is different from that of the base member. However, the material of the hydrophilic polymer layer may be the same as that of the base member as long as prescribed advantages can be obtained.

The polymer constituting the hydrophilic polymer layer is made of a hydrophilic material (e.g., hydrophilic polymer). However, an additive or the like other than the above material may be contained as long as it does not impair the hydrophilicity. The hydrophilic material means a material that can be dissolved by 0.0001 part by mass or more in 100 parts by mass of water at room temperature (20° C. to 23° C.). It is preferable that the hydrophilic material may be dissolved by 0.01 part by mass or more in 100 parts by mass of water, even preferably by 0.1 part by mass or more and further preferably by 1 part by mass or more.

It is preferable to use a hydrophilic polymer having a hydroxy group as the hydrophilic polymer. The use of a hydrophilic polymer having a hydroxy group is preferable because not only it is high in wettability but also enables formation of a surface having excellent antifouling property against body fluid. The hydrophilic polymer having a hydroxy group as mentioned above is preferably a polymer having an acidic hydroxy group. More specifically, the hydrophilic polymer having a hydroxy group is preferably a polymer having a group selected from a carboxyl group and a sulfonic acid group, most preferably a polymer having a carboxyl group. The carboxyl group or the sulfonic acid group may be in the form of a salt.

Examples of the hydrophilic polymer having a hydroxy group include polymethacrylic acid, polyacrylic acid, poly(vinylbenzoic acid), poly(thiophen-3-acetic acid), poly(4-styrenesulphonic acid), polyvinyl sulphonic acid, and poly(2-acrylamide-2-methylpropane sulfonic acid) and their salts. The above examples are homopolymers, and copolymers of hydrophilic monomers each constituting a hydrophilic polymer or copolymers of such a hydrophilic monomer and another monomer can be used preferably.

In the case where the hydrophilic polymer having a hydroxy group is a copolymer, the hydrophilic monomer constituting the copolymer is preferably a monomer having a group selected from an allyl group, a vinyl group, and a (meth)acryloyl group, most preferably a monomer having a (meth)acryloyl group. Preferable examples of such a monomer include (meth)acrylic acid, vinylbenzoic acid, styrenesulfonic acid, vinylsulfonic acid, and 2-acrylamide-2-methylpropane sulfonic acid and their salts. Among these examples, a monomer selected from (meth)acrylic acid and 2-acrylamide-2-methylpropane sulfonic acid and their salts is more preferable, a monomer selected from (meth)acrylic acid and its salts is the most preferable.

It is preferable that the hydrophilic polymer having a hydroxy group have an amide group in addition to a hydroxy group because not only such a hydrophilic polymer has high water wettability but also it can inhibit sticking of mucus and enables formation of a surface that can inhibit loss of cilia and excessive proliferation of goblet cells. Furthermore, such a hydrophilic polymer is preferable because such a hydrophilic polymer is also high in biocompatibility since it can inhibit sticking of mucus, and inhibit loss of cilia and excessive proliferation of goblet cells.

Examples of the acidic hydrophilic polymer having a hydroxy group and an amide group are polyamides having a carboxyl group and a copolymer of a monomer having a hydroxy group and a monomer having an amide group.

Preferable examples of the polyamides having a carboxyl group include polyamino acids such as polyaspartic acid and polyglutamic acid and polypeptides.

As the monomer having a hydroxy group, a monomer selected from methacrylic acid, acrylic acid, vinylbenzoic acid, thiophen-3-acetic acid, 4-styrenesulphonic acid, vinylsulphonic acid, and 2-acrylamide-2-methylpropane sulfonic acid and their salts can be used preferably.

From the viewpoint of the ease of polymerization, it is preferable to use, as the monomer having an amide group, a monomer selected from a monomer having a (meth)acrylamide group and N-vinylcarboxylic acid amide (including a cyclic one). Preferable examples of such a monomer include N-vinylpyrrolidone, N-vinylcaprolactam, N-vinylacetamide, N-methyl-N-vinylacetamide, N-vinylformamide, N,N-dimethylacrylamide, N,N-diethylacrylamide, N-isopropylacrylamide, N-(2-hydroxyethyl)acrylamide, acryloyl morpholine, and acrylamide. Among these monomers, from the viewpoint of inhibiting sticking of mucus, loss of cilia, and excessive proliferation of goblet cells, N-vinylpyrrolidone and N,N-dimethylacrylamide are preferable, and N,N-dimethylacrylamide is the most preferable.

Preferable examples of the hydrophilic polymer having an amide group in addition to a hydroxy group and being a copolymer include a (meth)acrylic acid/N-vinylpyrrolidone copolymer, a (meth)acrylic acid/N,N-dimethylacrylamide copolymer, a 2-acrylamide-2-methylpropane sulfonic acid/N-vinylpyrrolidone copolymer, and a 2-acrylamide-2-methylpropane sulfonic acid/N,N-dimethylacrylamide copolymer, and (meth)acrylic acid/N, N-dimethylacrylamide copolymer is the most preferable.

In the case of using a copolymer of a monomer having a hydroxy group and a monomer having an amide group, the copolymerization ratio, (mass of monomer having hydroxy group)/(mass of monomer having amide group), is preferably within a range of 1/99 to 99/1.

In copolymerization, the proportion of the monomer having a hydroxy group is more preferably 2 mass % or larger, even preferably 5 mass % or larger and further preferably 10 mass % or larger. In copolymerization, the proportion of the monomer having a hydroxy group is more preferably 90 mass % or smaller, even preferably 80 mass % or smaller and further preferably 70 mass % or smaller. In copolymerization, the proportion of the monomer having an amide group is more preferably 10 mass % or larger, even preferably 20 mass % or larger and further preferably 30 mass % or larger. In copolymerization, the proportion of the monomer having an amide group is more preferably 98 mass % or smaller, even preferably 95 mass % or smaller and further preferably 90 mass % or smaller.

In the case where the copolymerization ratio is within the above range, the function of inhibiting sticking of mucus and the functions of inhibiting loss of cilia and excessive proliferation of goblet cells are likely to be exhibited.

It is possible to further copolymerize the monomer having a hydroxy group and the monomer having an amide group with one or plural kinds of monomers selected from monomers having a different hydroxy group and/or amide group or monomers having neither a hydroxy group nor an amide group.

Preferable examples of monomers other than described above include hydroxyethyl (meth)acrylate, hydroxypropyl (meth)acrylate, hydroxybutyl (meth)acrylate, hydroxyethyl (meth)acrylamide, glycerol (meth)acrylate, caprolactone modified 2-hydroxyethyl (meth)acrylate, N-(4-hydroxyphenyl) maleimide, hydroxy styrene, and vinyl alcohol (carboxylic acid vinyl ester as a precursor). Among these monomers, from the viewpoint of the ease of polymerization, it is preferable to use a monomer having a (meth)acryloyl group, even preferably a (meth)acrylic acid ester monomer. Among the above monomers, from the viewpoints of inhibiting sticking of mucus, loss of cilia, and excessive proliferation of goblet cells, it is most preferable to use hydroxyethyl (meth)acrylate, hydroxypropyl (meth)acrylate, or glycerol (meth)acrylate, most preferably hydroxyethyl (meth)acrylate. It is also possible to use a monomer having such a feature as hydrophilicity, an antibacterial property, or an antifouling property. The hydrophilic polymer layer may include an additive or the like not mentioned above as long as it does not impair properties required for the stent. Furthermore, the hydrophilic polymer layer may include one or plural kinds of other hydrophilic polymers in addition to the hydrophilic polymer having a hydroxy group. However, since this tends to complicate a manufacturing method, the hydrophilic polymer layer is preferably made up of only one kind of hydrophilic polymer having a hydroxy group.

The term “one kind of polymer” means a polymer or a polymer group (isomer, complex, etc.) manufactured by one synthetic reaction. In the case where a copolymerized polymer is manufactured using plural monomers, polymers synthesized with different blending ratios are not regarded as the same polymer even if they employ the same kinds of monomers.

The expression “a hydrophilic polymer layer is made up of only one kind of hydrophilic polymer having a hydroxy group” means that the hydrophilic polymer layer contains no polymer other than the hydrophilic polymer having a hydroxy group, and if the hydrophilic polymer layer contains another polymer, the proportion of the other polymer with respect to 100 parts by mass of the hydrophilic polymer having a hydroxy group is more preferably 0.1 part by mass or smaller, even preferably 0.0001 part by mass or smaller.

In particular, in the case where the other polymer is an alkaline polymer, a problem in transparency arises if its content is larger than the above range. In related arts, both of an acidic polymer and an alkaline polymer are used because a hydrophilic polymer is laminated on the surface of a base member of a stent utilizing electrostatic absorption. In contrast, in an illustrative aspect of the present disclosure, the hydrophilic polymer layer made up of only one kind of polymer can be formed and fixed to the surface of the base member of the stent.

In the embodiment, the expression “a hydrophilic polymer layer having a hydroxy group is fixed to at least a part of the surface of the base member of the stent” means that the hydrophilic polymer layer is fixed to the surface of the base member of the stent by chemical bonds such as hydrogen bonds, ion bonds, van der Waals bonds, hydrophobic bonds, formation of complexes, or the like. The hydrophilic polymer layer may be bonded to the base member by covalent bonds. However, since this makes it difficult to manufacture the stent by a simple process, it is rather preferable that no covalent bonds are formed between the hydrophilic polymer layer and the base member.

In the embodiment (first embodiment), in order to inhibit sticking of mucus on the inside surface of the stent for a respiratory organ that has the inside surface and the outside surface, a hydrophilic polymer layer needs to be provided on at least a part of the inside surface of the stent, and the hydrophilic polymer layer is preferably provided on the entire inside surface. In another embodiment (second embodiment), in order to inhibit loss of cilia and excessive proliferation of goblet cells, a hydrophilic polymer layer need to be provided on at least a part of the outside surface of the stent, and the hydrophilic polymer layer is preferably provided on the entire outside surface. The hydrophilic polymer layer is even preferably provided on the inside surface and the outside surface of the stent, and the hydrophilic polymer layer is further preferably provided on the entire surfaces of the stent.

It is preferable that no covalent bonds are formed between the base member and the hydrophilic polymer layer because this makes it possible to manufacture the stent by a simple process. Whether no covalent bonds are formed is determined by judging whether no chemically reactive groups exist. Specific examples of chemically reactive groups include an azetidinium group, an epoxy group, an isocyanate group, an aziridine group, and an azlactone group and combinations thereof, but not limited to these.

The thickness of the layer including a hydrophilic polymer is preferably larger than or equal to 1 nm and smaller than 1000 nm when a cross section of a stent that is frozen in a water-retaining state (hereinafter referred to as a “frozen state”) is observed using a scanning transmission electron microscope. This is because when the thickness is within the range, the function of inhibiting sticking of mucus and the function of inhibiting loss of cilia and excessive proliferation of goblet cells are likely to be exhibited. The thickness of the layer including a hydrophilic polymer of the stent in a frozen state is more preferably 10 nm or larger, further preferably 20 nm or larger and most preferably 30 nm or larger. The thickness of the layer including a hydrophilic polymer of the stent in a frozen state is more preferably 900 nm or smaller, even preferably 800 nm or smaller and most preferably 700 nm or smaller. The thickness of the layer including a hydrophilic polymer of the stent in a frozen state can be measured by an observation using a scanning transmission electron microscope and a cryotransfer holder.

The thickness of the layer including a hydrophilic polymer of the stent in a dry state is preferably within a range of 1 nm to 1000 nm, because when the thickness is within this range, the function of inhibiting sticking of mucus and the function of inhibiting loss of cilia and excessive proliferation of goblet cells are likely to be exhibited. The thickness of the layer including a hydrophilic polymer of the stent in a dry state is more preferably 10 nm or larger, further preferably 20 nm or larger. The thickness of the layer including a hydrophilic polymer of the stent in a dry state is more preferably 900 nm or smaller, even preferably 800 nm or smaller and most preferably 700 nm or smaller.

As described above, the layer including a hydrophilic polymer is preferably separated into two or more layers or two or more phases.

The state in which the layer including a hydrophilic polymer is separated into two or more layers means a state in which a multilayer structure of two or more layers is observed in the layer including a hydrophilic polymer when a cross section of the stent is observed using a transmission electron microscope. In the case where it is difficult to judge whether the layer is separated merely by an observation using a transmission electron microscope, a judgment is made by analyzing elements or compositions in a cross section of the stent using a method capable of element analysis or composition analysis such as a scanning transmission electron microscopy, electron energy loss spectroscopy, energy-dispersive X-ray spectroscopy, or time-of-flight secondary ion mass spectrometry.

The state in which the layer including a hydrophilic polymer is phase-separated into two or more phases means a state in which a phase separation into two or more phases is observed in the layer including a hydrophilic polymer when a cross section of the stent is observed using a transmission electron microscope. In the case where it is difficult to make a phase separation judgment merely by an observation using a transmission electron microscope, a judgment is made in the same manner as described above.

Conventionally, to form a polymer layer including two or more layers or two or more phases, it is necessary to use two or more kinds of polymers. In contrast, in an illustrative aspect of the present disclosure, it has been found that a layer including a hydrophilic polymer that is separated into two or more layers or two or more phases can be formed on the surface of the base member even in the case where only one kind of polymer exists.

As described above, the stent according to the embodiment preferably include, between the base member and the hydrophilic polymer layer, a mixed layer in which the components of the base member and the components of the hydrophilic polymer layer are mixed together. In the case where the stent according to the embodiment includes the mixed layer, the layer including a hydrophobic polymer has a layered structure of two or more layers including the hydrophilic polymer layer and the mixed layer.

In the case where the layer including a hydrophobic polymer has a multilayer structure of two or more layers, the layer including a hydrophobic polymer is so thick that the function of inhibiting sticking of mucus and the function of inhibiting loss of cilia and excessive proliferation of goblet cells are enhanced. In the case where the layer including a hydrophobic polymer is phase-separated into two or more phases, it is easier to distinguish foreign matter such as dirt, dust or the like when a cross section of the stent is observed using a transmission electron microscope. Thus, it is easier to check whether a polymer layer has been formed on the surface of the base member of the stent, which enables an efficient quality inspection.

A state in which the components of the hydrophilic polymer layer and the components of the base member are mixed together can be checked by detecting elements originating from the base member in the mixed layer when a cross section of the stent is observed using an observation method capable of element analysis or composition analysis such as scanning transmission electron microscopy, electron energy loss spectroscopy, energy-dispersive X-ray spectroscopy, or time-of-flight secondary ion mass spectrometry. The hydrophilic polymer layer can be fixed to the base member more strongly by the mixing of the components of the hydrophilic polymer layer and the components of the base member.

In the case where the stent has a mixed layer in which the components of the hydrophilic polymer layer and the components of the base member are mixed together, it is preferable that a two-layer structure of the hydrophilic polymer layer and the mixed layer be observed. The thickness of the mixed layer is preferably 3% or more of the total thickness of the mixed layer and the hydrophilic polymer layer, even preferably 5% or more and further preferably 10% or more. The thickness of the mixed layer is preferably 98% or less of the total thickness of the mixed layer and the hydrophilic polymer layer, even preferably 95% or less and further preferably 90% or less, and most preferably 80% or less. A thickness ratio of the mixed layer being too small is not preferable because it means that the mixing of the hydrophilic polymer and the base member is insufficient. A thickness ratio of the mixed layer being too large is not preferable because the properties of the hydrophilic polymer layer may not be sufficiently exhibited.

The number of layers or phases of the layer including a hydrophilic polymer is preferably two or three because in this case the stent has high transparency, even preferably two.

The function of inhibiting sticking of mucus attained by an illustrative aspect of the present disclosure can be evaluated by a mucin sticking test that uses mucin extracted from saliva of human. A mucin sticking amount as obtained by this test being smaller is more preferable because this indicates the effect of inhibiting sticking of mucus more and high biocompatibility, such that the risks of infection through the stent and movement of the stent are lowered.

The stuck amount of mucin with respect to a silicone base member is preferably 50% or smaller, even preferably 40% or smaller and most preferably 30% or smaller. The details of a measurement method will be described later.

### <Manufacturing Method of Stent>

Next, a manufacturing method of the stent according to the embodiment will be described.

A stent according to the embodiment (first embodiment) can be manufactured by forming a hydrophilic polymer layer on at least a part of the inside surface of a base member. A stent according to another embodiment (second embodiment) can be manufactured by forming a hydrophilic polymer layer on at least a part of the outside surface of a base member.

In the first embodiment, a hydrophilic polymer layer is preferably formed on the entire inside surface of the base member, and a hydrophilic polymer layer may be formed on at least a part of the outside surface of the base member. In the second embodiment, a hydrophilic polymer layer is preferably formed on the entire outside surface of the base member, and a hydrophilic polymer layer may be formed on at least a part of the inside surface of the base member. A hydrophilic polymer layer is more preferably formed on the inside surface and the outside surface of the base member, and a hydrophilic polymer layer is further preferably formed on the entire surface of the base member. In the following description, a manufacturing method of the stent according to the first embodiment will be described in detail. However, the descriptions for the first embodiment except for the location of the hydrophilic polymer layer can be applied to the second embodiment.

A stent according to the embodiment can be manufactured by covering at least a part of the inside surface of the base member with a solution containing a hydrophilic polymer. There are no particular limitations on the covering method; a common method such as dipping, spraying, applying, or printing can be employed.

Among these methods, it is preferable to employ a method of heating the solution in a state where the base member is immersed in a solution containing a hydrophilic polymer having a hydroxy group. It is also possible to form a hydrophilic polymer layer on a part of the surface of the base member by spraying or applying a polymer solution onto or on a part of the surface of the base member. Furthermore, a hydrophilic polymer layer can be formed on a part of the surface of the base member by heating the solution in a state where only its inside surface is in contact with a polymer solution or only its outside surface is in contact with a polymer solution.

From the viewpoint of a manufacturing process, it is preferable to form a hydrophilic polymer layer on at least a part of the inside surface of a base member that has been shaped into a desired shape in advance.

To prevent movement of the stent after the stent is set and left, for example, a base member having a size and a shape that are suitable for the shape of a respiratory organ of a target patient may be used by generating data being high in anatomical accuracy by 3D-CT and using a 3D printing technique on the basis of an anatomical analysis.

The inventors have found that a hydrophilic polymer having a hydroxy group can be fixed on the surface of a base member of a stent and the stent is allowed to exhibit the function of inhibiting sticking of mucus and the function of inhibiting loss of cilia and excessive proliferation of goblet cells by a very simple method of adjusting the initial pH of a solution containing a hydrophilic polymer having a hydroxy group to 2.0 or larger and 6.0 or smaller, setting the base member of a stent in the solution, and heating the solution in this state, instead of a known special method such as a method utilizing electrostatic absorption in which both of an acidic polymer and an alkaline polymer are used. This is very important industrially from the viewpoint of shortening of a manufacturing process.

In the case where a polymer layer is formed on the surface of the base member of a stent using only one kind of a hydrophilic polymer having a hydroxy group, the related art has a problem that, because of an insufficient thickness of the layer, it is difficult to impart to the stent the function of inhibiting sticking of mucus sufficiently and the function of inhibiting loss of cilia and excessive proliferation of goblet cells.

In general, a polymer layer formed becomes thicker as the molecular weight of a hydrophilic polymer increases. However, the thickness of a polymer layer formed has an upper limit because too large a molecular weight may make the hydrophilic polymer more difficult to handle during manufacture because of increase in viscosity. In addition, in general, a polymer layer formed becomes thicker as the concentration of a hydrophilic polymer in a solution used in manufacture increases. However, in the case where the concentration of a hydrophilic polymer is too high, increased viscosity may make the hydrophilic polymer more difficult to handle during manufacture. Thus, the concentration of a hydrophilic polymer is limited in a manner similar to too large molecular weight.

However, in the case where the stent according to the embodiment includes the mixed layer, the layer including a hydrophilic polymer has a layered structure of two or more layers including a hydrophilic polymer layer and a mixed layer though only one kind of hydrophilic polymer having a hydroxy group is used. As a result, even when a hydrophilic polymer having a molecular weight within a range described below is used or a concentration of a hydrophilic polymer in a solution during manufacture is set in a range described below, the thickness of the layer including a hydrophilic polymer can be increased, which makes it easier to obtain a sufficient function of inhibiting sticking of mucus and a sufficient function of inhibiting loss of cilia and excessive proliferation of goblet cells.

The hydrophilic polymer having a hydroxy group used in the present disclosure preferably has a molecular weight of 2000 to 1,500,000. The molecular weight of the hydrophilic polymer having a hydroxy group is more preferably 5000 or larger, further preferably 10,000 or larger. The molecular weight of the hydrophilic polymer having a hydroxy group is more preferably 1,200,000 or smaller, further preferably 1,000,000 or smaller. A molecular weight used here is a mass average molecular weight in terms of polyethyleneglycol that is measured by gel permeation chromatography (aqueous solvent).

In general, the thickness of a hydrophilic polymer layer formed increases as the concentration of a hydrophilic polymer in a solution during manufacture increases. However, when the concentration of a hydrophilic polymer is too high, increase in viscosity may make its handling during manufacture more difficult. Thus, the concentration of a hydrophilic polymer having a hydroxy group is preferably within a range of 0.0001 to 30 mass %. The concentration of a hydrophilic polymer having a hydroxy group is more preferably 0.001 mass % or higher, further preferably 0.005 mass % or higher. The concentration of a hydrophilic polymer having a hydroxy group is more preferably 20% or lower, further preferably 15 mass % or lower.

In the above-described process, the initial pH of a solution containing a hydrophilic polymer is preferably within a range of 2.0 to 6.0 because in this range the solution does not become muddy and a highly transparent stent can be obtained. The initial pH is more preferably 2.2 or larger, further preferably 2.4 or larger, even further preferably 2.5 or larger, and most preferably 2.6 or larger. The initial pH is more preferably 5.0 or smaller, further preferably 4.5 or smaller, and most preferably 4.0 or smaller.

In the case where the initial pH is 2.0 or larger, the solution is less likely to become muddy. The solution being not muddy is preferable because in this state a living body tissue reaction tends to be found early during observation using an endoscope or the like. An initial pH value being larger than 6.0 is not preferable because in this case a hydrophilic polymer layer tends not to be formed so as to be separated into two or more layers or two or more phases and hence the function of inhibiting sticking of mucus and the function of inhibiting loss of cilia and excessive proliferation of goblet cells are lowered.

A pH value of a solution as described above can be measured using a pH meter (e.g., “Eutech pH2700” produced by Eutech Instruments Pte Ltd.). The initial pH of a solution containing a hydrophilic polymer having a hydroxy group means a pH value measured after adding the hydrophilic polymer fully to a solution and making the solution uniform by stirring it for 2 hours at room temperature (23 to 25° C.) using a rotor and before setting a base member and heating the solution. In the present disclosure, a pH value measured is rounded off to one decimal place.

The pH of a solution may vary by heating treatment. The pH of a solution after the heating treatment is preferably within a range of 2.0 to 6.5. The pH of a solution after heating is more preferably 2.2 or larger, further preferably 2.3 or larger, and most preferably 2.4 or larger. The pH of a solution after heating is more preferably 5.9 or smaller, further preferably 5.5 or smaller, even further preferably 5.0 or smaller, particularly preferably, and most preferably 4.5 or smaller.

In the case where the pH of a solution after the heating treatment is within the above range, a suitable pH condition can be obtained during the heating treatment, so that a stent obtained has preferable physical properties. The pH of a solution can be adjusted by performing neutralization treatment or adding water after the surface of a stent is modified by the heating treatment according to the present disclosure. However, the pH of a solution after the heating treatment here is a pH value of the solution before being subjected to such pH adjusting treatment.

Water is a preferable solvent of a solution described above containing a hydrophilic polymer having a hydroxy group. The pH of a solution containing a hydrophilic polymer can be adjusted by adding to the solution an acidic substance such as acetic acid, citric acid, formic acid, ascorbic acid, trifluoromethanesulfone acid, methanesulfone acid, nitric acid, sulfuric acid, phosphoric acid, or hydrochloric acid. Among these acidic substances, from the viewpoints of low volatility and high safety to a living body, citric acid, ascorbic acid, and sulfuric acid are preferable. It is preferable to add a buffer to a solution for fine pH adjustment.

Any known buffer that is physiologically compatible can be used as the above-mentioned buffer. Buffers that can be suitably used in the present disclosure are known to those skilled in the art. Examples of such buffers include boric acid, salts of boric acid (e.g., sodium borate), citric acid, salts of citric acid (e.g., potassium citrate), bicarbonates (e.g., sodium bicarbonate), a phosphoric acid buffer liquid (e.g., Na₂HPO₄, NaH₂PO₄, and KH₂PO₄), TRIS (tris(hyroxymethyl)aminomethane), 2-bis(2-hydroxyethyl)amino-2-(hydroxymethyl)-1,3-propanediol, bis-aminopolyol, triethanolamine, ACES (N-(2-acetoamide)-2-aminoethanesulfonic acid), BES (N,N-bis(2-hydroxyethyl)-2-aminoethanesulfonic acid), HEPES (4-(2-hydroxyethyl)-1-piperazineethane sulfonic acid), MES (2-(N-morpholino) ethanesulfonic acid), MOPS (3-[N-morpholino]-propanesulfonic acid), PIPES (piperazine-N,N′-bis(2-ethanesulfonic acid), and TES (N-[tris(hydroxymethyl)methyl]-2-aminoethanesulfonic acid), and their salts. Each of the above buffers is used in an amount that is necessary to make the buffer effective to attain a desired pH value. Usually, each buffer should exist in the above-mentioned solution at 0.001 to 2 mass %. It is preferable that each buffer exist at 0.01 to 1 mass %, even preferably 0.05 to 0.30 mass %. Each buffer may exist in a range from any of the above lower limits to any of the above upper limits.

Examples of heating methods include a high-pressure vapor sterilization method, electromagnetic wave (γ-ray, microwave, or the like) irradiation, a dry heat method, and a flame method. The high-pressure vapor sterilization method is the most preferable from the viewpoints of the function of inhibiting sticking of mucus, the function of inhibiting loss of cilia and excessive proliferation of goblet cells, and shortening of a manufacturing process. It is preferable to use an autoclave as an apparatus.

From the viewpoints of manufacturing a stent that is superior in the function of inhibiting sticking of mucus and the function of inhibiting loss of cilia and excessive proliferation of goblet cells and less affecting the strength of the stent itself, the heating temperature is preferably within a range of 60° C. to 200° C. The heating temperature is more preferably 80° C. or higher, further preferably 100° C. or higher, even further preferably 101° C. or higher, and most preferably 110° C. or higher. The heating temperature is more preferably 180° C. or lower, further preferably 170° C. or lower and most preferably 150° C. or lower.

The heating time is preferably within a range of 5 to 600 minutes because if the heating time is too short, a stent that is superior in the function of inhibiting sticking of mucus and the function of inhibiting loss of cilia and excessive proliferation of goblet cells cannot be obtained, and if the heating time is too long, the strength of a stent itself is affected. The heating time is more preferably 10 minutes or longer, further preferably 15 minutes or longer. The heating time is preferably 400 minutes or shorter, further preferably 300 minutes or shorter.

Another treatment may be further performed on the stent obtained after the above heating treatment. Examples of the other treatment include a method of subjecting the stent to similar heating treatment again in a solution containing a hydrophilic polymer having a hydroxy group, a method of subjecting the stent to similar heating treatment after replacing the solution with a solution not containing a hydrophilic polymer, a method of subjecting the stent to similar heating treatment again in a solution not containing a polymer, a method of applying radiation, a method of performing LbL treatment (layer by layer treatment) in which the base member is coated with polymer materials having opposite charge polarities alternately one layer at a time, a method of performing crosslinking treatment using metal ions, and a method of performing chemical crosslinking treatment. However, in view of the concept of the present disclosure of making the surface of a stent hydrophilic by a simple method, it is preferable to perform another treatment in such a range that the manufacturing process is not made too complex.

Radiation used for the above-described irradiation is preferably any kind of ion beams, an electron beam, a positron beam, an X-ray, a γ-ray, or a neutron beam. It is even preferable to use an electron beam or a γ-ray, most preferably a γ-ray.

As the above-mentioned LbL treatment, for example, it is preferable to use a treatment as described in WO 2013/024800 that uses an acidic polymer and an alkaline polymer.

The metal ion used in the above-mentioned crosslinking treatment using metal ions is preferably any kind of metal ions, even preferably monovalent and divalent metal ions and most preferably divalent metal ions. A chelate complex may also be used.

The above-mentioned chemical crosslinking treatment is preferably, for example, a reaction between an epoxide group and a carboxyl group as described in JP 2014-533381 (WO 2013/074535) or a known treatment for causing crosslinking with a suitable acidic hydrophilic polymer having a hydroxy group.

In the above-mentioned method of performing similar heating treatment after replacing the solution with a solution not containing a hydrophilic polymer, the solution not containing a hydrophilic polymer is not particularly limited. However, use of a buffer solution is preferable. The buffer may be any of the above-mentioned ones.

The pH of the buffer solution is preferably within a range of 6.3 to 7.8, which is physiologically allowable. The pH of the buffer solution is more preferably 6.5 or larger, further preferably 6.8 or larger. The pH of the buffer solution is preferably 7.6 or smaller, further preferably 7.4 or smaller.

To allow the stent to exhibit the function of inhibiting sticking of mucus and the function of inhibiting loss of cilia and excessive proliferation of goblet cells while functioning as a stent for a respiratory organ, the ratio X:Y between the thickness X of the layer including a hydrophilic polymer layer and the thickness Y of the base member is preferably within a range of 1:400 to 1:120,000, even preferably 1:800 to 1:100,000, further preferably 1:1200 to 1:80,000, and particularly preferably 1:1500 to 1:60,000.

The shape of the stent according to the embodiment will be described.

FIGS. 1, 3, and 4 are schematic views of the stents according to the embodiment.

Although there are no particular limitations on the shape of the stent according to the embodiment, the stent may include a tubular structure portion as shown in FIG. 1. As shown in FIG. 4, the shape of the stent according to the embodiment may have branches. The stent according to the embodiment is preferably shaped so as to conform to the shape of a respiratory organ to which the stent is applied.

There are no particular limitations on the size of the stent according to the embodiment. However, to allow the stent to function as a stent for a respiratory organ, the outer diameter of the tubular structure portion is preferably 4 mm or larger and 24 mm or smaller, and the thickness of the stent is preferably 0.2 mm or larger and 2 mm or smaller. The outer diameter of the tubular structure portion is more preferably 6 mm or larger and 20 mm or smaller, and the thickness of the stent is more preferably 0.25 mm or larger and 1.5 mm or smaller.

The term “outer diameter” as used herein is defined so that projections or projections/recesses are included in the case where they are formed on the outer circumferential surface. In the case where no projections or no projections/recesses are formed on the outer circumferential surface, the “outer diameter” is defined so that no projections or no projections/recesses are included and it is sufficient that a portion whose outer diameter is within the above range exist in a part of the stent.

As mentioned above, there are no particular limitations on the shape of the stent according to the embodiment. However, to prevent movement after the stent is set and left, it is preferable that the outside surface of the stent be formed with a projection(s) or a projection(s)/recess(es). It is preferable that plural projections or projections/recesses be formed.

Plural projections or plural projections/recesses may be arranged either regularly or randomly.

In the case where projections are formed on the outside surface of the stent, plural projections 40A may be arranged regularly or randomly as shown in FIGS. 1, 3, and 4, for example.

Projections or projections/recesses may be arranged locally on the outside surface, be arranged on the entire outside surface, or dot the outside surface.

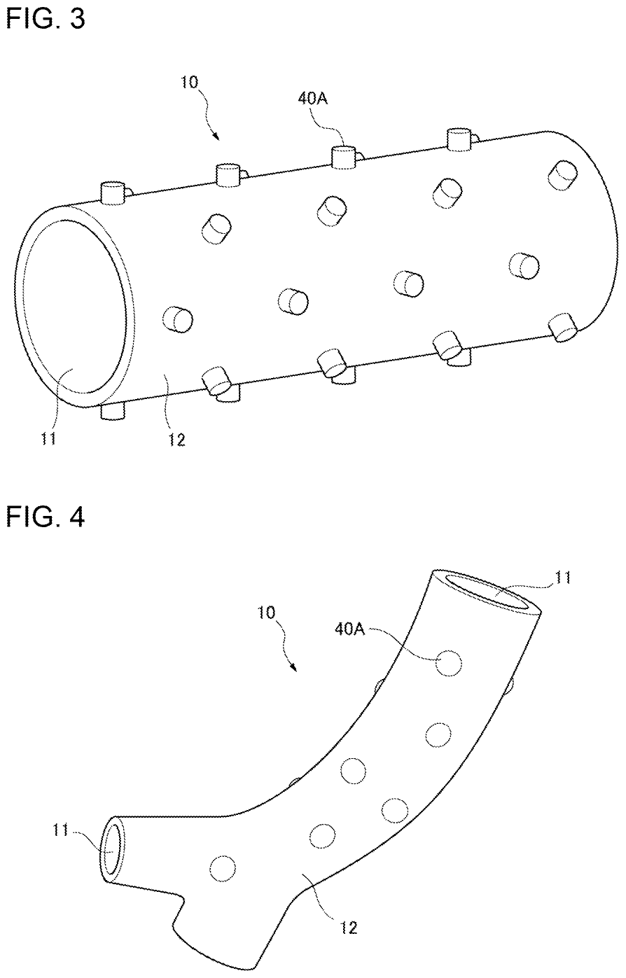

There are no particular limitations on the shape of the projections 40A; each of them may be shaped like a semisphere, a cylinder, a cone, a prism, a polygonal spindle, a hook, or the like. More specifically, for example, each of the projections 40A may be shaped like a semisphere as shown in FIG. 1 or a cylinder as shown in FIG. 3.

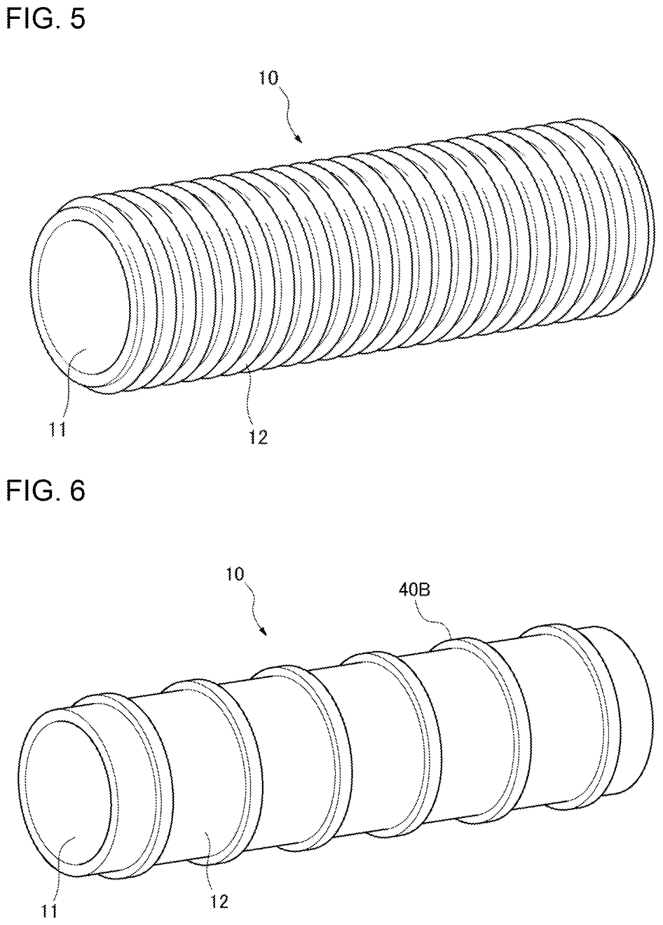

There are no particular limitations on the shape of the projections/recesses; they may be shaped like folds, embossed shapes, certain patterns (e.g., lines, waves, or stars) or the like. More specifically, for example, the projections/recesses may be shaped like folds as shown in FIG. 5 or line-shaped patterns like projections 40B of a stent shown in FIG. 6.

In the case where projections or projections/recesses are arranged on the outside surface of the stent, there are no particular limitations on the size of the projections. From the viewpoint of inhibiting stimuli to tissue, the size of the projections is preferably 4 mm or shorter, even preferably 3 mm or shorter and further preferably 2 mm or shorter.

To allow the stent to exhibit the function of preventing movement of itself after the stent is set and left, the size of the projections is preferably 0.1 mm or larger, even preferably 0.2 mm or larger.

For example, to prevent movement of the stent after being set and left, a stent having a size and a shape that are suitable for the shape of a respiratory organ of a patient to which the stent is to be applied may be produced by generating data being high in anatomical accuracy by 3D-CT and using a 3D printing technique on the basis of an anatomical analysis. The difference between the outer diameter of the stent and the inner diameter of a respiratory organ to which the stent is to be applied is preferably 10% or smaller, even preferably 8% or smaller, further preferably 6% or smaller, and particularly preferably 5% or smaller.

### <Method for Securing Air Flow by Relieving Stenosis of Respiratory Organ>

A method for securing air flow by relieving stenosis of a respiratory organ according to the embodiment is a method for securing air flow by relieving stenosis of a respiratory organ using a stent, the stent having an inside surface and an outside surface, for a respiratory organ, in which: the stent includes a base member and a hydrophilic polymer layer; the hydrophilic polymer layer contains a hydrophilic polymer having a hydroxy group and an amide group; and the hydrophilic polymer layer is provided on at least a part of the inside surface.

Because of the use of the above-described stent, the method for securing air flow by relieving stenosis of a respiratory organ according to the embodiment can inhibit sticking of mucus and is high in biocompatibility, and thus can inhibit occurrence of complications.

The above description regarding the stent can be cited as it is for the method for securing air flow by relieving stenosis of a respiratory organ according to the embodiment.

### INDUSTRIAL APPLICABILITY

The stent according to the embodiment is a stent for a respiratory organ, and for example, the stent can be applied to the respiratory tract, oral cavity, nasal tracts, pharynx, trachea, bronchi, bronchioles, and lungs. The stent according to the embodiment can also be applied to uses for securing a lumen by implanting the stent into a constricted part of various body cavities or vascular ducts (vascular system, esophagus, gastrointestinal tract, colon and small intestine, bile duct, pancreatic duct, lung pipes, ureter, nasal cavities and respiratory tract, trachea, bronchi, etc.) other than respiratory organs.

### EXAMPLES

An illustrative aspect of the present disclosure will be described below in detail using Inventive Examples and Comparative Examples. However, the present disclosure is not limited to them.

### Measurement Example 1: Water Wettability (Liquid Film Retention Time)

A stent was cleaned lightly at room temperature (23° C. to 25° C.) in 100 mL of a phosphoric acid buffer liquid in a beaker and then immersed in 100 mL of a new phosphoric acid buffer liquid for 24 hours or more. The stent was lifted up from the phosphoric acid buffer liquid, a time for which a liquid film was maintained on the surface of the stent being held in the air was measured visually, and an average time of three measurements was judged according to the following criteria.
A: The surface liquid film was maintained for 60 seconds or longer.
B: The surface liquid film was separated when a time of 30 seconds or longer and shorter than 60 seconds elapsed.
C: The surface liquid film was separated when a time of 5 seconds or longer and shorter than 30 seconds elapsed.
D: The surface liquid film was separated when a time of shorter than 5 seconds elapsed.

### Measurement Example 2: Measurement of Weight-Average Molecular Weight

A weight-average molecular weight of a hydrophilic polymer used was measured under the following conditions.

### (GPC Measurement Conditions)

Instrument: “Prominence GPC system” produced by Shimadzu Corporation
Pump: LC-20AD
Autosampler: SIL-20AHT
Column oven: CTO-20A
Detector: RID-10A
Column: GMPWXL produced by Tosoh Corporation (inner diameter 7.8 mm×30 cm; particle diameter 13 μm)
Solvent: water/methanol=1/1 (with 0.1 N lithium nitrate added)
Flow rate: 0.5 mL/min
Measurement time: 30 minutes
Sample concentration: 0.1 mass %
Injection amount: 100 μL
Standard sample: polyethylene oxide standard sample (0.1 kD to 1258 kD) produced by Agilent Technologies, Inc.

### Measurement Example 3: Initial pH Measurement Method

A pH value of a solution was measured using a pH meter “Eutech pH2700” produced by Eutech Instruments Pte Ltd. In the table, an initial pH value of a solution containing a hydrophilic polymer having a hydroxy group was a pH value that was measured after all of the hydrophilic polymer was added to a solution described in each of Examples and then the solution was made uniform by stirring it using a rotor for two hours at room temperature 23° C. to 25° C.).

### Measurement Example 4: Judgment as to Separation of Layer Including Hydrophilic Polymer

Whether a layer including a hydrophilic polymer is separated into two or more layers was judged by observing a cross section of a stent using a transmission electron microscope.
Instrument: transmission electron microscope “H-7100FA” produced by Hitachi, Ltd.
Acceleration voltage: 100 kV Sample preparation: Silicone-based base member (RuO₄-dyed ultrathin section method)
Hydrogel-based base member (OsO₄-dyed ultrathin section method or RuO₄-dyed ultrathin section method)

### Measurement Example 5: Elemental Composition Analysis of Layer Including Hydrophilic Polymer

Elemental composition analysis of a layer including a hydrophilic polymer was performed by analyzing a cross section of a stent that was frozen in a water-containing state using a cryotransfer holder by a transmission scanning electron microscope method and electron energy loss spectroscopy.
Instrument: field emission electron microscope (“JEM-2100F” produced by JEOL Ltd.)
Acceleration voltage: 200 kV
Measurement temperature: about −100° C.
Electron energy loss spectroscopy: GATAN GIF Tridiem
Image acquisition: Digital Micrograph
Sample preparation: RuO₄-dyed freezing ultrathin section method

### Measurement Example 6: Thickness of Layer Including Hydrophilic Polymer

A thickness of a layer including a hydrophilic polymer in a dry state was measured by observing a cross section of a stent in a dry state using a transmission electron microscope. A measurement was conducted under the conditions described in the above (Measurement example 4: judgment as to separation of layer including hydrophilic polymer). Thickness values at 35 positions in total were measured by measuring thickness values at five positions in each of fields of view of seven different places. An average value of the measured thickness values is shown in Table 1.

A thickness of a layer including a hydrophilic polymer in a frozen state was measured by observing a cross section of a stent that was frozen in a water-containing state using a cryotransfer holder using a transmission scanning electron microscope. A measurement was conducted under the conditions described in the above (Measurement example 5: elemental composition analysis of layer including hydrophilic polymer). Thickness values at 35 positions in total were measured by measuring thickness values at five positions in each of fields of view of seven different places. An average value of the measured thickness values is shown in Table 1.

### Measurement Example 7: In Vitro Mucus Sticking Test

Mucin was purified from saliva and a mucin solution having a concentration of 100 μg/mL was prepared. A stent was punched into disc-shaped pieces having a diameter of 4 mm, and then the obtained pieces were set in 48 respective wells of a microtiter plate. To each well, 600 μL of the mucin solution having a concentration of 100 g/mL was added and incubated at 37° C. for 20-24 hours. As a control, PBS was added instead of a mucin solution and incubated at 37° C. for 20-24 hours. After cleaning was performed three times using PBS, a blocking buffer (ThermoFisher Scientific 37570) was added and incubated at room temperature (23° C. to 25° C.) for one hour. After cleaning was performed three times using PBS, WGA (Biotinylated Wheat Germ Agglutinin (WGA), Vector Laboratories B-1025-5; diluted 500 times by PBS) was added and incubated at room temperature for one hour. After cleaning was performed three times using PBS, horseradish peroxidase (HRP)-conjugated streptavidin (HRP-Streptavidin, Sigma-Aldrich RABHRP3-600UL) was added and incubated at room temperature for one hour. After cleaning was performed three times using PBS, 250 μL of a solution of TMB (3, 3′, 5, 5′-tetramethylbenzidine (TMB) substrate, Thermo Scientific PI34028) was added and incubated at room temperature for 15 to 30 minutes. Then each sample was taken out, 250 μL of 2M sulfuric acid was added and absorbance at 450 nm was measured using a microplate spectrophotometer. A mucus (mucin) sticking amount was calculated according to the following formula (1).

$$( Mucus sticking amount ( \% ) ) = ( A s - A s b ) \times 100 / ( Ac - Acb ) \qquad ( 1 )$$

As: absorbance of a sample
Asb: absorbance of a blank solution (PBS is used instead of a mucin solution and incubated for one night) for the sample
Ac: absorbance of a stent made of silicone (Dumon stent)
Acb: absorbance of a blank solution (PBS is used instead of a mucin solution and incubated for one night) for the stent made of silicone (Dumon stent)

### Measurement Example 8: Evaluation of Biocompatibility by Test of Implanting Respiratory Tract Stents into Pig Bronchi

Referring to the paper of H. S. Jung et al. (Scientific Reports 11, 7958, 2021) and other papers, respiratory tract stents having a length of 1 cm were left in the bronchi of a pig and the surfaces of the respiratory tract stents thus left were observed regularly using a bronchus endoscope. Two respiratory tract stents were left for one pig (one stent in the left bronchus and the other in the right bronchus).

After four weeks from the leaving, mucus existing on the surface of each respiratory tract stent and around it was collected and the mass of the mucus was measured.

Furthermore, after four weeks from the leaving, the respiratory tract stents were removed and samples were produced by subjecting them to H. E. dyeing (Hematoxylin Eosin dyeing) or PAS dyeing (Periodic Acid-Schiff dyeing). The samples thus obtained were observed using a microscope and the degree of loss of cilia and the number of goblet cells are evaluated in the form of scores according to the following criteria.

### Degree of Loss of Cilia (H. E. Dyeing):

No loss: 0
Low: 1
Medium: 2
High: 3

### Goblet Cells (PAS Dyeing):

0 to 20%: 0
larger than 20% and 60% or smaller: 1
larger than 60% and 80% or smaller: 2
larger than 80% and 90% or smaller: 3
larger than 90% and 100% or smaller: 4

### [Phosphoric Acid Buffer Liquid (PBS)]

The composition of a phosphoric acid buffer liquid that was used in processes of the following Inventive Examples and Comparative Examples and the above-described measurements was as follows.
KCl: 0.2 g/L
KH₂PO₄: 0.2 g/L
NaCl: 8.0 g/L
Na₂HPO₄ (anhydrous): 1.15 g/L
EDTA: 0.25 g/L

### Referential Example 1

A silicone stent base member (see FIG. 5) having an outer diameter of 14 mm, a thickness of 1 mm, and a length of 4 cm was formed using liquid silicone rubber (“SILASTIC (trademark) 3D 3335 LSR” produced by The Dow Chemical Company) for a 3D printer and a 3D printer “L320” produced by German RepRap GmbH.

### Inventive Example 1

The silicone stent base member of Referential Example 1 was put into a solution obtained by adding citric acid to an aqueous solution in which an acrylic acid/N,N-dimetylacrylamide copolymer (copolymerization mole ratio: 1/9, Mw: 800,000; produced by Osaka Organic Chemical Industry Ltd.) was contained in pure water at 0.2 mass % to regulate the pH to 2.8, and the solution was heated at 121° C. for 30 minutes using an autoclave. A resulting stent was cleaned by a phosphoric acid buffer liquid, dried naturally, and evaluated by the above-described methods. Results are shown in Table 1.

As a result of analysis for an elemental composition of a layer including a hydrophilic polymer according to the above-described measurement example 5, one layer was a mixed layer of the components of the base member and the components of the hydrophilic polymer layer and the other layer was a layer made up of only the hydrophilic polymer.

### Inventive Example 2

The silicone stent base member of Referential Example 1 was put into a solution obtained by adding citric acid to a solution in which an acrylic acid/N,N-dimetylacrylamide copolymer (copolymerization mole ratio: 1/9, Mw: 700,000; produced by Osaka Organic Chemical Industry Ltd.) was contained in a phosphoric acid buffer liquid at 0.2 mass % to regulate the pH to 2.6, and the solution was heated at 121° C. for 30 minutes using the autoclave. A resulting stent was cleaned by a phosphoric acid buffer liquid, dried naturally, and evaluated by the above-described methods. Results are shown in Table 1.

### Inventive Example 3

The silicone stent base member of Referential Example 1 was put into a solution obtained by adding citric acid to an aqueous solution in which an acrylic acid/N,N-dimetylacrylamide copolymer (copolymerization mole ratio: 1/9, Mw: 800,000; produced by Osaka Organic Chemical Industry Ltd.) was contained in pure water at 0.2 mass % to regulate the pH to 2.5, and the solution was heated at 121° C. for 30 minutes using the autoclave. A resulting stent was cleaned by a phosphoric acid buffer, dried naturally, and evaluated by the above-described methods. Results are shown in Table 1.

### Inventive Example 4

A silicone stent base member of Referential Example 1 was put into a solution obtained by adding citric acid to a solution in which an acrylic acid/vinylpyrrolidone copolymer (copolymerization mole ratio: 1/4, Mw: 500,000; produced by Osaka Organic Chemical Industry Ltd.) was contained in a phosphoric acid buffer liquid at 0.1 mass % to regulate the pH to 3.2, and the solution was heated at 121° C. for 30 minutes using the autoclave. A resulting stent was cleaned by a phosphoric acid buffer liquid, dried naturally, and evaluated by the above-described methods. Results are shown in Table 1.

### Inventive Example 5

A silicone stent (Dumon stent) base member was put into a solution obtained by adding citric acid to a solution in which an acrylic acid/N,N-dimetylacrylamide copolymer (copolymerization mole ratio: 1/9, Mw: 800,000; produced by Osaka Organic Chemical Industry Ltd.) was contained in a phosphoric acid buffer liquid at 0.2 mass % to regulate the pH to 3.3, and the solution was heated at 121° C. for 30 minutes using the autoclave. A resulting stent was cleaned by a phosphoric acid buffer liquid, dried naturally, and evaluated by the above-described methods. Results are shown in Table 1.

### Inventive Example 6

A silicone stent (Dumon stent) base member was put into a solution obtained by adding citric acid to a solution in which an acrylic acid/N,N-dimetylacrylamide copolymer (copolymerization mole ratio: 1/9, Mw: 500,000; produced by Osaka Organic Chemical Industry Ltd.) was contained in a phosphoric acid buffer liquid at 0.2 mass % to regulate the pH to 3.0, and the solution was heated at 121° C. for 30 minutes using the autoclave. A resulting stent was cleaned by a phosphoric acid buffer liquid, dried naturally, and evaluated by the above-described methods. Results are shown in Table 1.

### Inventive Example 7

A silicone stent (Dumon stent) base member was put into a solution obtained by adding citric acid to a solution in which an acrylic acid/N,N-dimetylacrylamide copolymer (copolymerization mole ratio: 1/2, Mw: 700,000; produced by Osaka Organic Chemical Industry Ltd.) was contained in a phosphoric acid buffer liquid at 0.2 mass % to regulate the pH to 3.1, and the solution was heated at 121° C. for 30 minutes using the autoclave. A resulting stent was cleaned by a phosphoric acid buffer liquid, dried naturally, and evaluated by the above-described methods. Results are shown in Table 1.

### Inventive Example 8

A silicone stent (Dumon stent) base member was put into a solution obtained by adding citric acid to a solution in which an acrylic acid/vinylpyrrolidone copolymer (copolymerization mole ratio: 1/4, Mw: 800,000; produced by Osaka Organic Chemical Industry Ltd.) was contained in a phosphoric acid buffer liquid at 0.1 mass % to regulate the pH to 4.1, and the solution was heated at 121° C. for 30 minutes using the autoclave. A resulting stent was cleaned by a phosphoric acid buffer liquid, dried naturally, and evaluated by the above-described methods. Results are shown in Table 1.

### Inventive Example 9

A silicone stent (Dumon stent) base member was put into a solution obtained by adding citric acid to a solution in which an acrylic acid/vinylpyrrolidone copolymer (copolymerization mole ratio: 1/9, Mw: 400,000; produced by Osaka Organic Chemical Industry Ltd.) was contained in a phosphoric acid buffer liquid at 0.1 mass % to regulate the pH to 4.3, and the solution was heated at 121° C. for 30 minutes using the autoclave. A resulting stent was cleaned by a phosphoric acid buffer liquid, dried naturally, and evaluated by the above-described methods. Results are shown in Table 1.

### Inventive Example 10

A silicone stent (Dumon stent) base member was put into a solution obtained by adding citric acid to a solution in which an acrylic acid/N,N-dimetylacrylamide copolymer (copolymerization mole ratio: 1/9, Mw: 500,000; produced by Osaka Organic Chemical Industry Ltd.) was contained in a phosphoric acid buffer liquid at 0.2 mass % to regulate the pH to 2.7, and the solution was heated at 121° C. for 30 minutes using the autoclave. A resulting stent was cleaned by a phosphoric acid buffer liquid, dried naturally, and evaluated by the above-described methods. Results are shown in Table 1.

### Inventive Example 11

A silicone stent (Dumon stent) base member was put into a solution obtained by adding citric acid to a solution in which an acrylic acid/N,N-dimetylacrylamide copolymer (copolymerization mole ratio: 1/9, Mw: 800,000; produced by Osaka Organic Chemical Industry Ltd.) was contained in a phosphoric acid buffer liquid at 0.2 mass % to regulate the pH to 2.9, and the solution was heated at 121° C. for 30 minutes using the autoclave. A resulting stent was cleaned by a phosphoric acid buffer liquid, dried naturally, and evaluated by the above-described methods. Results are shown in Table 1.

### Comparative Example 1

A silicone stent (Dumon stent) base member was cleaned by a phosphoric acid buffer liquid, dried naturally, and evaluated by the above-described methods. Results are shown in Table 1.

### Comparative Example 2

A silicone stent base member of Referential Example 1 was cleaned by a phosphoric acid buffer liquid, dried naturally, and evaluated by the above-described methods. Results are shown in Table 1.

### Comparative Example 3

A silicone stent (Dumon stent) base member was put into a solution obtained by adding citric acid to a solution in which an acrylic acid/N,N-dimetylacrylamide copolymer (copolymerization mole ratio: 1/2, Mw: 500,000; produced by Osaka Organic Chemical Industry Ltd.) was contained in a phosphoric acid buffer liquid at 0.03 mass % to regulate the pH to 3.2, and the solution was heated at 121° C. for 30 minutes using the autoclave. A resulting stent was cleaned by a phosphoric acid buffer liquid, dried naturally, and evaluated by the above-described methods. Results are shown in Table 1.

### Comparative Example 4

A silicone stent base member of Referential Example 1 was put into a solution obtained by adding citric acid to a solution in which an acrylic acid/vinylpyrrolidone copolymer (copolymerization mole ratio: 1/4, Mw: 500,000; produced by Osaka Organic Chemical Industry Ltd.) was contained in a phosphoric acid buffer liquid at 0.03 mass % to regulate the pH to 4.5, and the solution was heated at 121° C. for 30 minutes using the autoclave. A resulting stent was cleaned by a phosphoric acid buffer liquid, dried naturally, and evaluated by the above-described methods. Results are shown in Table 1.

### Inventive Example 12

The stent obtained in Inventive Example 8 was evaluated by the method described in Measurement example 8. Results are shown in Table 2.

### Inventive Example 13

The stent obtained in Inventive Example 11 was evaluated by the method described in Measurement example 8. Results are shown in Table 2.

### Comparative Example 5

The stent obtained in Comparative Example 1 was evaluated by the method described in Measurement example 8. Results are shown in Table 2.

### Comparative Example 6

The stent obtained in Comparative Example 3 was evaluated by the method described in Measurement example 8. Results are shown in Table 2.

### Inventive Example 14

A silicone stent (Dumon stent) base member was put into a solution obtained by adding citric acid to a solution in which an acrylic acid/N,N-dimetylacrylamide copolymer (copolymerization mole ratio: 1/9, Mw: 800,000; produced by Osaka Organic Chemical Industry Ltd.) was contained in a phosphoric acid buffer liquid at 0.2 mass % to regulate the pH to 2.9, and the solution was heated at 121° C. for 30 minutes using the autoclave. A resulting stent was cleaned by a phosphoric acid buffer liquid, dried naturally, and heated again in a phosphoric acid buffer liquid at 121° C. for 30 minutes using the autoclave. A resulting stent was cleaned by a phosphoric acid buffer liquid, dried naturally, and evaluated by the above-described methods. Results are shown in Table 1.

### Inventive Example 15

A solution was obtained by adding citric acid to a solution in which an acrylic acid/N,N-dimetylacrylamide copolymer (copolymerization mole ratio: 1/9, Mw: 800,000; produced by Osaka Organic Chemical Industry Ltd.) was contained in a phosphoric acid buffer liquid at 0.2 mass % to regulate the pH to 2.9. A silicone stent (Dumon stent) base member was put into the solution such that only the outside surface of the base member was in contact to the solution, and the solution was heated at 121° C. for 30 minutes using the autoclave. A resulting stent was cleaned by a phosphoric acid buffer liquid, dried naturally, and evaluated by the above-described methods. Results are shown in Table 1.

### Inventive Example 16

The stent obtained in Inventive Example 14 was evaluated by the method described in Measurement example 8. Results are shown in Table 2.

### Inventive Example 17

The stent obtained in Inventive Example 15 was evaluated by the method described in Measurement example 8. Results are shown in Table 2.

The layer including a hydrophilic polymer of each of the stents obtained by Inventive Examples 1-11, 14, 15 and Comparative Examples 3 and 4 was subjected to the elemental composition analysis by the method described in Measurement example 5. As a result, it was found that one layer was a mixed layer of the components of the base member and the components of the hydrophilic polymer layer and the other layer was made up of only the hydrophilic polymer.

TABLE 1

| | | | Concentration | Weight- | Liquid |
|---|---|---|---|---|---|
| | | | of hydrophilic | average | film |
| | Base | | polymer | molecular | retention |
| | member | Hydrophilic polymer | (mass %) | weight | time |
| Inventive | Referential | acrylic acid/N,N- | 0.2 | 800,000 | A |
| Ex. 1 | Example 1 | dimetylacrylamide copolymer | | | |
| Inventive | Referential | acrylic acid/N,N- | 0.2 | 700,000 | B |
| Ex. 2 | Example 1 | dimetylacrylamide copolymer | | | |
| Inventive | Referential | acrylic acid/N,N- | 0.2 | 800,000 | B |
| Ex. 3 | Example 1 | dimetylacrylamide copolymer | | | |
| Inventive | Referential | acrylic acid/vinylpyrrolidone | 0.1 | 500,000 | A |
| Ex. 4 | Example 1 | copolymer | | | |
| Inventive | Dumon | acrylic acid/N,N- | 0.2 | 800,000 | A |
| Ex. 5 | stent | dimetylacrylamide copolymer | | | |
| Inventive | Dumon | acrylic acid/N,N- | 0.2 | 500,000 | A |
| Ex. 6 | stent | dimetylacrylamide copolymer | | | |
| Inventive | Dumon | acrylic acid/N,N- | 0.2 | 700,000 | A |
| Ex. 7 | stent | dimetylacrylamide copolymer | | | |
| Inventive | Dumon | acrylic acid/vinylpyrrolidone | 0.1 | 800,000 | A |
| Ex. 8 | stent | copolymer | | | |
| Inventive | Dumon | acrylic acid/vinylpyrrolidone | 0.1 | 400,000 | B |
| Ex. 9 | stent | copolymer | | | |
| Inventive | Dumon | acrylic acid/N,N- | 0.2 | 500,000 | A |
| Ex. 10 | stent | dimetylacrylamide copolymer | | | |
| Inventive | Dumon | acrylic acid/N,N- | 0.2 | 800,000 | A |
| Ex. 11 | stent | dimetylacrylamide copolymer | | | |
| Comp. | Dumon | — | — | — | D |
| Ex. 1 | stent | | | | |
| Comp. | Referential | — | — | — | D |
| Ex. 2 | Example 1 | | | | |
| Comp. | Dumon | acrylic acid/N,N- | 0.03 | 500,000 | C |
| Ex. 3 | stent | dimetylacrylamide copolymer | | | |
| Comp. | Referential | acrylic acid/vinylpyrrolidone | 0.03 | 500,000 | C |
| Ex. 4 | Example 1 | copolymer | | | |
| Inventive | Dumon | acrylic acid/N,N- | 0.2 | 800,000 | A |
| Ex. 14 | stent | dimetylacrylamide copolymer | | | |
| Inventive | Dumon | acrylic acid/N,N- | 0.2 | 800,000 | A |
| Ex. 15 | stent | dimetylacrylamide copolymer | | | |

| | | | | Thickness | Ratio between | |
|---|---|---|---|---|---|---|
| | | Number of | | of layer | thickness X of | |
| | | layers | Mucin | including | layer including | Re-heating |
| | | including | sticking | hydrophilic | hydrophilic polymer | treatment by |
| | Initial | hydrophilic | amount | polymer | and thickness Y of | autoclave after |
| | pH | polymer | (%) | (nm) | stent | polymer coating |
| Inventive | 2.8 | 2 | 25 | 21 | 1:47619 | — |
| Ex. 1 | | | | | | |
| Inventive | 2.6 | 2 | 38 | 17 | 1:58824 | — |
| Ex. 2 | | | | | | |
| Inventive | 2.5 | 2 | 36 | 19 | 1:52632 | — |
| Ex. 3 | | | | | | |
| Inventive | 3.2 | 2 | 39 | 18 | 1:55556 | — |
| Ex. 4 | | | | | | |
| Inventive | 3.3 | 2 | 22 | 34 | 1:44118 | — |
| Ex. 5 | | | | | | |
| Inventive | 3.0 | 2 | 33 | 26 | 1:57692 | — |
| Ex. 6 | | | | | | |
| Inventive | 3.1 | 2 | 23 | 31 | 1:48387 | — |
| Ex. 7 | | | | | | |
| Inventive | 4.1 | 2 | 38 | 29 | 1:51724 | — |
| Ex. 8 | | | | | | |
| Inventive | 4.3 | 2 | 44 | 24 | 1:62500 | — |
| Ex. 9 | | | | | | |
| Inventive | 2.7 | 2 | 41 | 21 | 1:71429 | — |
| Ex. 10 | | | | | | |
| Inventive | 2.9 | 2 | 20 | 32 | 1:46875 | — |
| Ex. 11 | | | | | | |
| Comp. | — | — | 100 | — | — | — |
| Ex. 1 | | | | | | |
| Comp. | — | — | 97 | — | — | — |
| Ex. 2 | | | | | | |
| Comp. | 3.2 | 2 | 58 | 7 | 1:214286 | — |
| Ex. 3 | | | | | | |
| Comp. | 4.5 | 2 | 56 | 8 | 1:125000 | — |
| Ex. 4 | | | | | | |
| Inventive | 2.9 | 2 | 15 | 23 | 1:65217 | Done |
| Ex. 14 | | | | | | |
| Inventive | 2.9 | 2 | 22 | 30 | 1:50000 | — |
| Ex. 15 | | | | | | |

TABLE 2

| | Mucus weight | Score of loss | Score of number of |
|---|---|---|---|
| | (mg) | of cilia | goblet cells |
| Inventive Ex. 12 | 110 | 1.6 | 2.5 |
| Inventive Ex. 13 | 80 | 1.4 | 2.2 |
| Comp. Ex. 5 | 250 | 2.1 | 3.6 |
| Comp. Ex. 6 | 230 | 2.0 | 3.5 |
| Inventive Ex. 16 | 70 | 1.3 | 2.1 |
| Inventive Ex. 17 | 180 | 1.6 | 2.3 |

### REFERENCE SIGNS LIST

10 stent
11 inside surface
12 outside surface
21 base member
22 mixed layer
23 hydrophilic polymer layer
40A projection
40B projection

1. A stent for a respiratory organ, the stent having an inside surface and an outside surface, wherein:
the stent comprises a base member and a hydrophilic polymer layer;
the hydrophilic polymer layer contains a hydrophilic polymer having a hydroxy group and an amide group; and
the hydrophilic polymer layer is provided on at least a part of the inside surface.

2. A stent for a respiratory organ, the stent having an inside surface and an outside surface, wherein:
the stent comprises a base member and a hydrophilic polymer layer;
the hydrophilic polymer layer contains a hydrophilic polymer having a hydroxy group and an amide group; and
the hydrophilic polymer layer is provided on at least a part of the outside surface.

3. The stent according to claim 1, wherein the base member contains a silicone resin.

4. The stent according to claim 1, comprising a mixed layer of a component of the base member and a component of the hydrophilic polymer layer, the mixed layer being disposed between the base member and the hydrophilic polymer layer.

5. The stent according to claim 1, wherein the ratio X:Y between a thickness X of a layer including the hydrophilic polymer and a thickness Y of the base member is within a range of 1:400 to 1:120,000.

6. The stent according to claim 1, comprising a tubular structure portion.

7. The stent according to claim 6, wherein the tubular structure portion has an outer diameter of 4 mm or larger and 24 mm or smaller and has a thickness of 0.2 mm or larger and 2 mm or smaller.

8. The stent according to claim 1, comprising plural projections or projections and recesses on the outside surface.

9. The stent according to claim 1, wherein the respiratory organ is a trachea, bronchus, or lung.

10. The stent according to claim 1, wherein the hydrophilic polymer having a hydroxy group and an amide group is at least one polymer selected from the group consisting of polyamides having a carboxyl group and copolymers of a monomer having a hydroxy group and a monomer having an amide group.

11. The stent according to claim 10, wherein the monomer having a hydroxy group is at least one monomer selected from the group consisting of methacrylic acid, acrylic acid, vinylbenzoic acid, thiophen-3-acetic acid, 4-styrenesulphonic acid, vinylsulphonic acid, 2-acrylamide-2-methylpropane sulfonic acid, and their salts.

12. The stent according to claim 10, wherein the monomer having an amide group is at least one monomer selected from the group consisting of N-vinylpyrrolidone, N-vinylacetamide, N,N-dimethylacrylamide, N,N-diethylacrylamide, N-isopropylacrylamide, N-(2-hydroxyethyl)acrylamide, and acrylamide.

13. A method for securing air flow by relieving stenosis of a respiratory organ using a stent for a respiratory organ, the stent having an inside surface and an outside surface, wherein:
the stent comprises a base member and a hydrophilic polymer layer;
the hydrophilic polymer layer contains a hydrophilic polymer having a hydroxy group and an amide group; and
the hydrophilic polymer layer is provided on at least a part of the inside surface.

14. A method for securing air flow by relieving stenosis of a respiratory organ using a stent for a respiratory organ, the stent having an inside surface and an outside surface, wherein:
the stent comprises a base member and a hydrophilic polymer layer;
the hydrophilic polymer layer contains a hydrophilic polymer having a hydroxy group and an amide group; and
the hydrophilic polymer layer is provided on at least a part of the outside surface.
